(19) United States
(12) Patent Application Publication
(10) Pub. No.: US 2025/0205412 A1
(43) Pub. Date: Jun. 26, 2025

(54) DISTRIBUTED NEGATIVE PRESSURE WOUND THERAPY SYSTEMS AND METHODS

(71) Applicant: T.J.Smith and Nephew,Limited, Hull (GB)

(72) Inventor: Felix Clarence Quintanar, Hull (GB)

(21) Appl. No.: 18/848,362

(22) PCT Filed: Mar. 30, 2023
(86) PCT No.: PCT/EP2023/058336

§ 371 Date: Sep. 18, 2024

(30) Foreign Application Priority Data
Apr. 4, 2022 (GB) 2204887.0

(51) Int. Cl. A61M 1/00; G16H 20/40; G16H 40/67

(52) U.S. Cl. CPC A61M 1/90; G16H 20/40; G16H 40/67; A61M 2205/18; A61M 2205/3327; A61M 2205/3331; A61M 2205/3553; A61M 2205/3561; A61M 2205/3592; A61M 2205/50; A61M 2230/63

(57) ABSTRACT

A negative pressure wound therapy system can include a negative pressure wound therapy device configured to provide, via a fluid flow path, negative pressure in accordance with at least one parameter to a wound of a patient covered by a wound dressing and measure pressure in the fluid flow path. The system can include one or more remote computing devices configured to communicate with the device via a low latency, high speed communication protocol, receive measured pressure in the fluid flow path from the device, based on processing the measured pressure, determine whether an adjustment to the at least one parameter of negative pressure is needed, and in response to determining that the adjustment is needed, communicate to the device a request to perform the adjustment, thereby causing the device to perform the adjustment in response to receiving the request.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

A negative pressure wound therapy system can include at least one negative pressure wound therapy device configured to provide, via a fluid flow path, negative pressure in accordance with at least one parameter to a wound of a patient covered by a wound dressing. The at least one negative pressure wound therapy device can be configured to measure pressure in the fluid flow path. The system can include one or more remote computing devices configured to communicate with the at least one negative pressure wound therapy device via a low latency, high speed communication protocol. The one or more remote computing devices can be configured to receive measured pressure in the fluid flow path from the at least one negative pressure wound therapy device. The one or more remote computing device can be configured to, based on processing the measured pressure, determine whether an adjustment to the at least one parameter of negative pressure is needed. The one or more remote computing device can be configured to, in response to determining that the adjustment is needed, communicate to the at least one negative pressure wound therapy device a request to perform the adjustment, wherein, the at least one negative pressure wound therapy device is configured to perform the adjustment in response to receiving the request. The at least one negative pressure wound therapy device can be configured to switch between different communication protocols responsive to detection of movement of the patient.

The negative pressure wound therapy system of any of the preceding paragraphs and/or any of the systems, devices, or apparatuses disclosed herein can include one or more of the following features. The low latency, high speed communication protocol can be a 4G LTE or 5G cellular communication protocol. The at least one negative pressure wound therapy device can be configured to switch from NB-IoT mode to LTE-M mode responsive to detection of movement of the patient and switch from LTE-M mode to NB-IoT mode responsive to lack of detection of movement of the patient. The adjustment can include at least one of changing a negative pressure set point, providing indication of abnormal operating condition, or pausing provision of negative pressure. The negative pressure wound therapy system can be configured to switch from a low speed, low energy consumption communication protocol to the low latency, high speed communication protocol responsive to the detection of movement of the patient.

The negative pressure wound therapy system of any of the preceding paragraphs and/or any of the systems, devices, or apparatuses disclosed herein can include one or more of the following features. The one or more remote computing devices can be configured to communicate to the at least one negative pressure wound therapy device a negative pressure wound therapy prescription. The at least one negative pressure wound therapy device can be configured to provide negative pressure in accordance with the negative pressure wound therapy prescription. The at least one negative pressure wound therapy device can be configured to provide negative pressure in accordance with the negative pressure wound therapy prescription responsive to verification of the negative pressure wound therapy prescription by a healthcare provider at the at least one negative pressure wound therapy device. The at least one negative pressure wound therapy device can include a plurality of negative pressure wound therapy devices configured to be controlled by the one or more remote computing devices. In response to detecting a failure in communication via the low latency, high speed communication protocol, communication with the at least one negative pressure wound therapy device can be performed using a different communication protocol via another negative pressure wound therapy device or another computing device. The different communication protocol can include a short range wireless communication protocol.

A negative pressure wound therapy device can include a negative pressure source configured to provide, via a fluid flow path, negative pressure in accordance with at least one parameter of therapy to a wound of a patient covered by a wound dressing. The device can include a pressure sensor configured to measure pressure in the fluid flow path. The device can include a communication circuitry configured to transmit data to and receive data from one or more remote computing devices via a low latency, high speed communication protocol. The communication circuitry can be configured to transmit pressure in the fluid flow path measured by the pressure sensor, thereby causing the one or more remote computing devices to determine an adjustment to the at least one parameter of therapy. The communication circuitry can be configured to receive from the one or more remote computing devices the adjustment of the at least one parameter of therapy. The communication circuitry can be configured to adjust the at least one parameter of therapy.

The negative pressure wound therapy device of any of the preceding paragraphs and/or any of the devices, systems, or apparatuses disclosed herein can include one or more of the following features. The low latency, high speed communication protocol can be a 4G LTE or 5G cellular communication protocol. The communication circuitry can be configured to switch between different communication protocols responsive to detection of movement of the patient. The communication circuitry can be configured to switch from a low speed, low energy consumption communication protocol to the low latency, high speed communication protocol responsive to the detection of movement of the patient. The communication circuitry can be configured to switch from NB-IoT mode to LTE-M mode responsive to detection of movement of the patient and switch from LTE-M mode to NB-IoT mode responsive to lack of detection of movement of the patient. The adjustment can include at least one of changing a negative pressure set point, providing indication of abnormal operating condition, or pausing provision of negative pressure by the negative pressure source. The communication circuitry can be configured to receive from the one or more remote computing devices a negative pressure wound therapy prescription. The negative pressure source can be configured to provide negative pressure in accordance with the negative pressure wound therapy prescription. The negative pressure source can be configured to provide negative pressure in accordance with the negative pressure wound therapy prescription responsive to verification of the negative pressure wound therapy prescription by a healthcare provider at the negative pressure wound therapy device.

A negative pressure wound therapy system can include at least one negative pressure wound therapy device configured to provide, via a fluid flow path, negative pressure in accordance with at least one parameter to a wound of a patient covered by a wound dressing. The at least one negative pressure wound therapy device can be configured to measure pressure in the fluid flow path. The system can include a non-transitory computer readable medium storing instructions that, when executed by one or more processors of one or more remote computing devices, cause the one or more processors to communicate with the at least one negative pressure wound therapy device via a low latency, high speed communication protocol. The one or more processors can be caused to receive measured pressure in the fluid flow path from the at least one negative pressure wound therapy device. The one or more processors can be caused to, based on processing the measured pressure, determine whether an adjustment to the at least one parameter of negative pressure is needed. The one or more processors can be caused to, in response to determining that the adjustment is needed, communicate to the at least one negative pressure wound therapy device a request to perform the adjustment, wherein, the at least one negative pressure wound therapy device is configured to perform the adjustment in response to receiving the request. The at least one negative pressure wound therapy device can be configured to switch between different communication protocols responsive to detection of movement of the patient.

The negative pressure wound therapy system of any of the preceding paragraphs and/or any of the systems, devices, or apparatuses disclosed herein can include one or more of the following features. The low latency, high speed communication protocol can be a 4G LTE or 5G cellular communication protocol. The negative pressure wound therapy system can be configured to switch from a low speed, low energy consumption communication protocol to the low latency, high speed communication protocol responsive to the detection of movement of the patient. The at least one negative pressure wound therapy device can be configured to switch from NB-IoT mode to LTE-M mode responsive to detection of movement of the patient and switch from LTE-M mode to NB-IoT mode responsive to lack of detection of movement of the patient. The adjustment can include at least one of changing a negative pressure set point, providing indication of abnormal operating condition, or pausing provision of negative pressure.

The negative pressure wound therapy system of any of the preceding paragraphs and/or any of the systems, devices, or apparatuses disclosed herein can include one or more of the following features. The one or more processors can be caused to communicate to the at least one negative pressure wound therapy device a negative pressure wound therapy prescription. The at least one negative pressure wound therapy device can be configured to provide negative pressure in accordance with the negative pressure wound therapy prescription. The at least one negative pressure wound therapy device can be configured to provide negative pressure in accordance with the negative pressure wound therapy prescription responsive to verification of the negative pressure wound therapy prescription by a healthcare provider at the at least one negative pressure wound therapy device. The at least one negative pressure wound therapy device can include a plurality of negative pressure wound therapy devices configured to be controlled by the one or more remote processors. In response to detecting a failure in communication via the low latency, high speed communication protocol, communication with the at least one negative pressure wound therapy device can be performed using a different communication protocol via another negative pressure wound therapy device or another computing device. The different communication protocol can include a short range wireless communication protocol.

A kit can include the system or device of any of the preceding paragraphs and/or any of the devices, systems, or apparatuses disclosed herein and a canister. A kit can include the system or device of any of the preceding paragraphs and/or any of the devices, systems, or apparatuses disclosed herein and the wound dressing.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a user interface.

FIG. 3 illustrates a schematic of a control system of a negative pressure wound therapy device.

FIG. 4 illustrates another negative pressure wound therapy system.

FIG. 5 illustrates a distributed negative pressure wound therapy system.

FIG. 6 illustrates a schematic of a control system of a connected negative pressure wound therapy device.

FIG. 7 illustrates a distributed medical device system.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as-X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than −X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

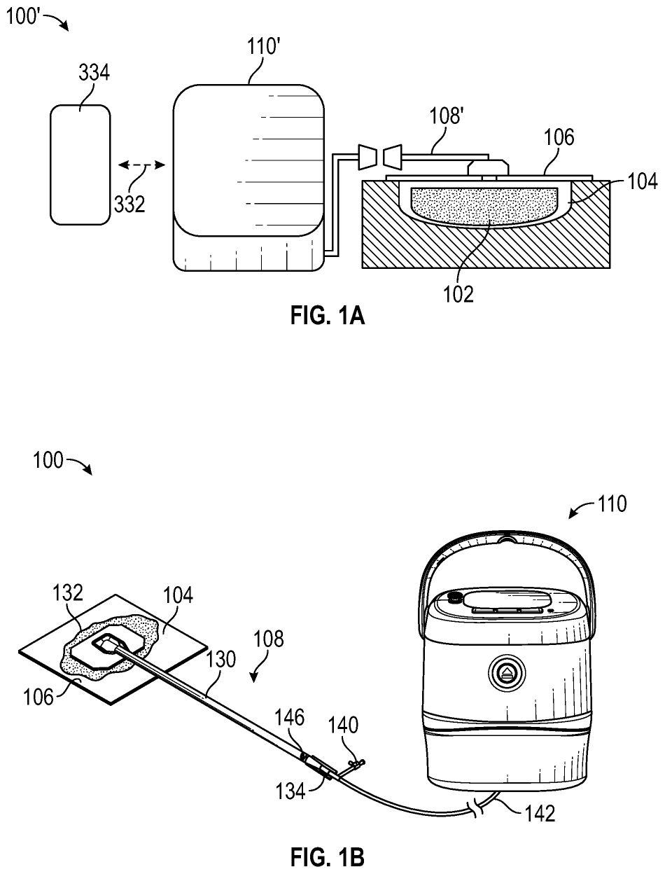

FIG. 1A schematically illustrates a negative pressure wound treatment system 100′ (sometimes referred to as a reduced or negative pressure wound therapy system, a TNP system, or a wound treatment system). In any implementations disclosed herein, though not required, the negative pressure wound treatment system 100′ can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108′ (also referred to herein as a flexible suction adapter or a fluidic connector) can be used to connect the wound cover 106 with a wound therapy device 110′ (sometimes as a whole or partially referred to as a “pump assembly”) configured to supply reduced or negative pressure. The conduit 108′ can be a single or multi lumen tube. A connector can be used to removably and selectively couple a conduit or tube of the device 110′ with the conduit 108′.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110′ and the wound cover 106, so as to supply the reduced pressure provided by the wound therapy device 110′ to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110′. In some cases, though not required, the wound therapy device 110′ can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110′ can operate with or without the use of an exudate canister. In some cases, as is illustrated, the wound therapy device 110′ can include an exudate canister. In some cases, configuring the wound therapy device 110′ and conduit 108′ so that the conduit 108′ can be quickly and easily removed from the wound therapy device 110′ can facilitate or improve the process of wound dressing or pump changes, if necessary. Any of the pump assemblies disclosed herein can have any suitable connection between the conduit 108′ and the pump.

The wound therapy device 110′ can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110′.

As will be described in greater detail below, the negative pressure wound treatment system 100′ can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be one or more of a smartphone, a tablet, a laptop or another standalone computer, a server (such as, a cloud server), another pump device, or the like. In some cases, the negative pressure wound treatment system 110′ can communicate with one or more cloud computing devices directly or via the device 334, which can be a smartphone, a tablet, a laptop or another standalone computer.

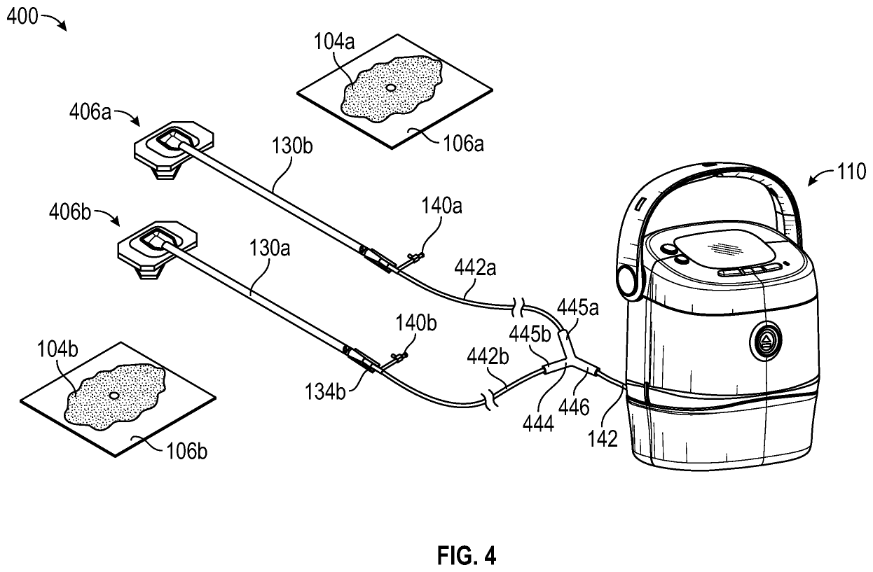

FIG. 1B illustrates another negative pressure wound treatment system 100. The negative pressure wound treatment system 100 can have any of the components, features, or other details of any of the other negative pressure wound treatment system disclosed herein, including without limitation the negative pressure wound treatment system 100′ illustrated in FIG. 1A or the negative pressure wound treatment system 400 illustrated in FIG. 4, in combination with or in place of any of the components, features, or other details of the negative pressure wound treatment system 100 shown in FIG. 1B and/or described herein. The negative pressure wound treatment system 100 can have a wound cover 106 over a wound 104 that can seal the wound 104. A conduit 108, such as a single or multi lumen tube can be used to connect the wound cover 106 with a wound therapy device 110 (sometimes as a whole or partially referred to as a “pump assembly”) configured to supply reduced or negative pressure. The wound cover 106 can be in fluidic communication with the wound 104.

With reference to FIG. 1B, the conduit 108 can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108 can be a Soft Port manufactured by Smith & Nephew. As mentioned, the negative pressure wound treatment system 100′ can include a source of negative pressure, such as the wound therapy device 110, capable of supplying negative pressure to the wound 104 through the conduit 108. Though not required, the wound therapy device 110 can also include a canister or other container for the storage of wound exudates and other fluids that can be removed from the wound.

The wound therapy device 110 can be connected to the connector 134 via a conduit or tube 142. In use, the applicator 132 can be placed over an aperture formed in a wound cover 106 that is placed over a suitably-prepared wound or wound 104. Subsequently, with the wound therapy device 110 connected via the tube 142 to the connector 134, the wound therapy device 110 can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge portion 130 and can each preferably comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970.®, or Gehring 879.®) or a nonwoven material, or terry-woven or loop-pile materials. The fibers may not necessarily be woven, and can include felted and flocked (including materials such as Flotex.®) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge portion 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108 can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. Preferably, the air leak 146 is located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International Publication No. WO2021/069642, each of which is incorporated by reference in its entirety as if fully set forth herein. In some cases, other suitable wound dressings can be utilized.

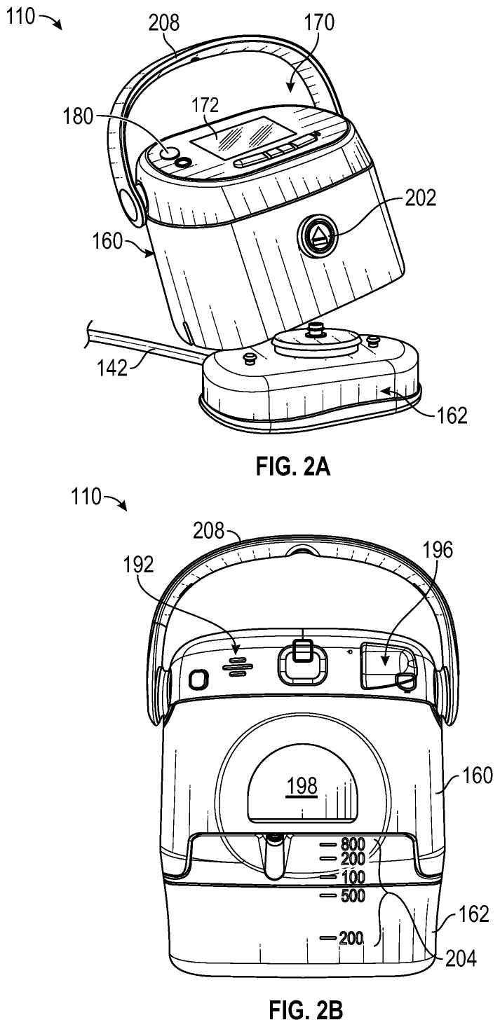

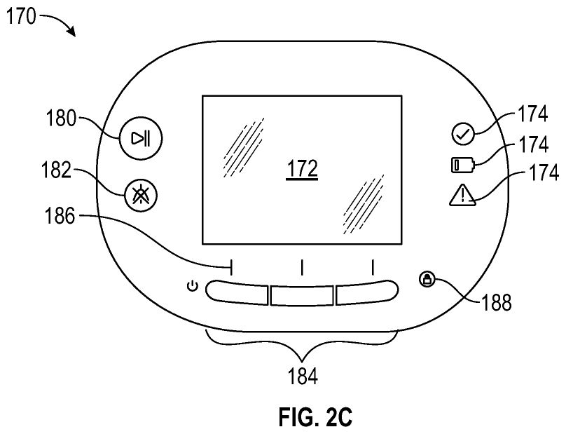

FIGS. 2A-2C show the negative pressure wound therapy device 110. As illustrated, a pump assembly 160 and canister 162 can be connected, thereby forming the wound therapy device 110. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and or a status of the pump. For example and without limitation, the one or more indicators 174 can be configured to provide a visual indication of a status of the pump assembly 160 or other components of the negative pressure wound treatment system 100, including without limitation the conduit 108 or the wound cover 106 (such as, to provide an indication of normal operation or abnormal operation, such as low battery, leak, canister full, blockage, overpressure, or the like). Any one or more suitable indicators can be additionally or alternatively used, such as visual, audio, tactile indicator, and so on.

FIG. 2B shows a back or rear view of the wound therapy device 110 shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108 in order to form a fluid flow path. The canister 162 can be replaced with another canister, such as when the canister 162 has been filled with fluid. With reference to FIG. 2A, the wound therapy device 110 can include a canister inlet tube 142 (also referred to herein as a dressing port connector) in fluid communication with the canister 162. For example and without limitation, the canister inlet tube 142 can be used to connect with the conduit 108.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110 can have a handle 208 that can be used to lift or carry the wound therapy device 110. The handle 208 can be coupled with the pump assembly 160 and can be rotatable relative to the wound therapy device 110 so that the handle can be rotated upward for lifting or carrying the wound therapy device 110 or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110.

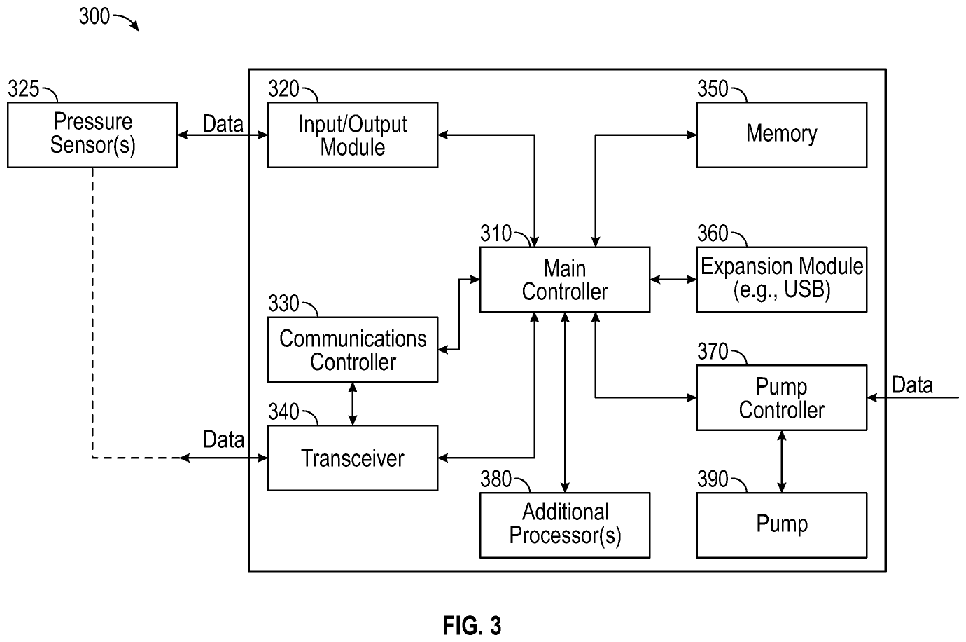

FIG. 3 illustrates a schematic of a control system 300 that can be employed in any of the wound therapy devices described herein, such as in the wound therapy device 110. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controllers 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110. The wound therapy device 110 can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110 to the first wound dressing 406a. As a non-limiting example, the first fluid flow path can include the path from the wound dressing 406a to the wound therapy device 110 or the path from the first wound dressing 406a to an inlet 446 of a branching attachment (or connector) 444 in fluidic connection with the wound therapy device 110. Similarly, a second fluid flow path can include components providing fluidic connection from the wound therapy device 110 to the second wound dressing 406b.

The system 400 can be similar to the system 100 with the exception that multiple wounds 104a and 140b are being treated by the system 400. The system 400 can include any one or more of the components of the system 100, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110 via a plurality of suction adapters, such as the adapter 108. The suction adapters can include any one or more of the components of the adapter 108, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110 can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the wound dressings 406a, 406b. Once all conduits and dressing components are coupled and operably positioned, the wound therapy device 110 can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110 can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850, International Publication No. WO2018/167199, International Publication No. WO2018/167199, or International Patent Application No. PCT/EP2022/079091, each of which is incorporated by reference in its entirety.

### Distributed Negative Pressure Wound Therapy

Existing negative pressure wound therapy devices tend to be stand-alone, non-connected devices. Such devices typically incorporate some sensing and processing capabilities to deliver negative pressure wound therapy to a patient. However, due to their stand-alone nature, such devices can be expensive, bulky, and error-prone.

At least some of the disclosed implementations relate to distributed negative pressure wound therapy systems that include reduced feature negative pressure devices (sometimes referred to as distributed negative pressure wound therapy devices or distNPWT) that provide therapy and communicate with one or more remote computing devices (which can be in the cloud) configured to process data sensed by the negative pressure devices and control the devices. For example, once negative pressure wound therapy prescription has been accepted by a negative pressure device, the device would provide therapy, and any adjustments to the therapy would be performed responsive to commands received the one or more remote computing devices. A distributed negative pressure wound therapy system sometime is referred to as a distNPWT system. One or more remote computing devices can function as a centralized decision maker configured to one or more of determine therapy, prescribe therapy, or adjust therapy. High speed and low latency communication protocols can be used for communication between the devices and the one or more remote computing devices. Such communication protocols can include cellular protocols, for example, 4G LTE, 5G, 6G, or higher generation. Advantages of at least some of the disclosed distNPWT systems include simplicity, cost effectiveness, ease of use, improved performance, reduction of errors, environmental friendliness, and disposability.

Therapy set points, alerts, alarms, device technical data, patient mobility, therapy logs, patient vital signs, patient area wireless sensing data (such as, Bluetooth or Bluetooth Low Energy), ambient and environment in use, or the like can be processed in real time (or substantially in real time) remotely and privately by the one or more remote computing devices (which sometimes can be referred to as a central control monitoring system). Real time remote patient sensing, tracking, and processing allows to create complex therapy procedures, such as flowcharts of therapy macros. The central control monitoring system can dynamically change the therapy parameters according to various patient-related parameters, such as sleep, exercise, daily activity, pain management, or the like.

The central control monitoring system can perform patient data processing and provide fast response time to determine patient events, alerts, alarms, therapy parameter changes, or the like for controlling distNPWT devices. Broadcasting patient data for medical professional care (which can be remote), digital animation of patient data (such as, vital signs), or wound healing determination can be achieved. Digital animation of patient data and remote patient care through distNPWT devices can be done with health care servers behaving and clustered as a supercomputer, connected to a gathered file system to run complex decision processes and simulations. Multiple redundant, cloud-based servers and applications may be ideal to implement multistage processing mechanisms to manage patient data safely and non-disruptively.

The centralization of controlling distNPWT devices could be one or more machine-to-machine interconnected super computers and cloud servers with redundancies and encrypted private communication protocols. The type of central control monitoring system could be done at hospital level, regional health level with various hospitals, country level preserving regulation and in compliance with any applicable laws or regulations (such as, HIPPA, GDPR, etc.), remote home healthcare care regions, or the like.

The central control monitoring system can use cloud server supercomputer infrastructure for 5G technology (or higher generation) with mobile edge distNPWT or other processing and application. Computer and storage services within communications service providers' datacenters at the edge of the 5G (or higher generation) network, secure carrier communication running the telecommunications network can be utilized. Latency can be avoided by reducing traffic across the Internet to reach distNPWT devices, enabling patients and medical professionals to take full advantage of the latency and bandwidth benefits offered by 5G (or higher) networks.

Tracking of wound healing status or infection, video feedback, audio feedback, patient vital signs, or the like can be remotely monitored and controlled by a medical professional, such as a doctor or nurse. Real time supervision of patient care, such as dressing changes, wound cleaning, setting or replacing the distNPWT device or components (such as, canisters), and supply management can be performed.

In some implementations, a distributed negative pressure wound therapy system can operate as an Internet of Things (IoT) system with distributed negative pressure wound therapy devices acting as IoT devices.

The central control monitoring system can be configured to process patient data and quickly determine (for instance, using machine learning) adjustment of therapy (or determine that no adjustment is needed) and instruct distNPWT devices to take appropriate action with no (or little) latency. Advantageously, the speed of processing and control can be one of the distinguishing differences of distNPWT systems.

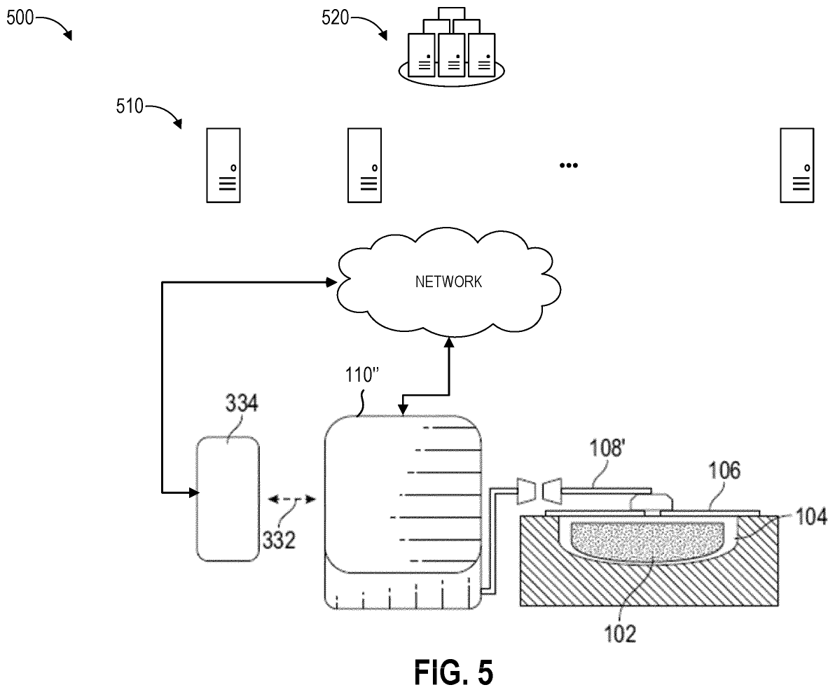

FIG. 5 illustrates a distributed negative pressure wound therapy system 500. The system 500 can include distributed (or cloud) computing devices 510 and 520 (which can form a central control monitoring system) connected to a distributed negative pressure wound therapy device 110″ via a network (such as, the Internet). The wound therapy device 110″ can be similar to any of the wound therapy devices described herein, such as 110′ or 110. Computing devices 510 can be edge computing devices. Computing devices 520 can be cloud computing devices. As described herein, the wound therapy device 110″ can communicate with the cloud computing devices via 4G LTE, 5G, 6G, or higher generation protocol. The communication can be direct or via the computing device 334.

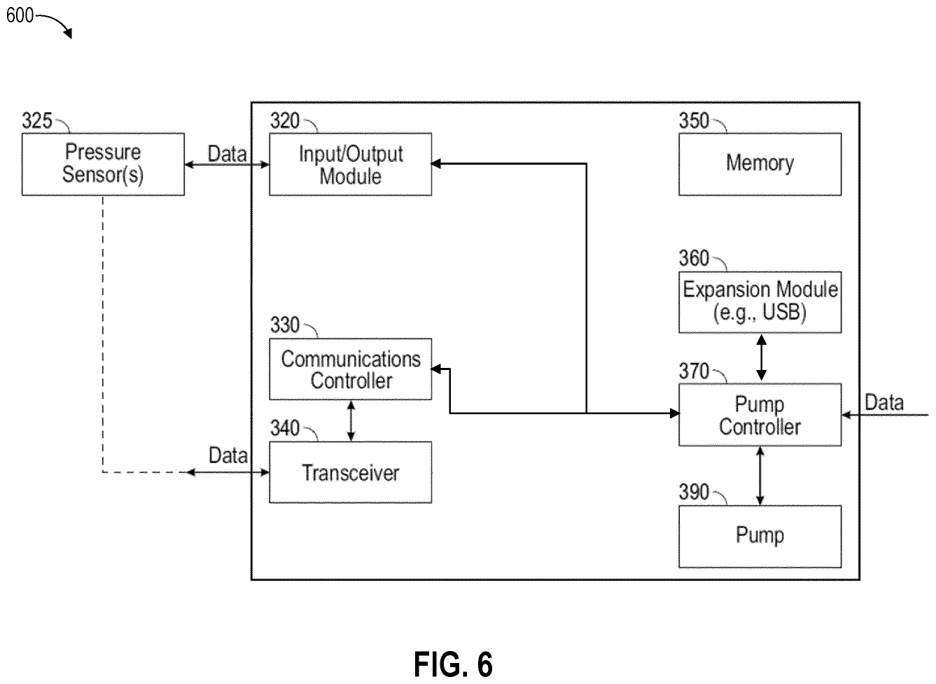

FIG. 6 illustrates a schematic of a control system 600 that can be employed by the wound therapy device 110″. Control system 600 can be similar to the control system 300 described herein with the exception of the following. Main controller 310 and additional processors 380 can be omitted from the control system 600. In addition to controlling the operation of the pump 390, pump controller 370 can provide basic control of the wound therapy device 110″, including interfacing with one or more of the input/output module 320, communication controller 330, or one or more explanation modules 360. The communications controller 330 and one or more transceivers 340 can facilitate communication with the computing devices 510 and 520 using 4G LTE, 5G, 6G, or higher generation protocols.

In some implementations, a negative pressure wound therapy prescription is communicated by the central control monitoring system to a distributed negative pressure wound therapy device. The prescription can be verified locally by a healthcare provider at the distributed negative pressure wound therapy device using any of the approaches described in U.S. Patent Publication No. 2021/0379272, which is incorporated by reference in its entirety. Subsequently, the distributed negative pressure wound therapy device can provide therapy in accordance with the prescription. For example, verification can be performed using a camera (which can be external or be integrated into the distributed negative pressure wound therapy device), via a password, via a biometric check, or the like. Verification can be advantageous to ensure that the correct distributed negative pressure wound therapy device (from a plurality of such devices) is being controlled.

The distributed negative pressure wound therapy device may rely on the central control monitoring system to perform the majority of processing and control tasks related to provision of therapy (with the exception of actual control of the negative pressure source, which can be performed by the device). The negative pressure wound therapy device can collect data, including data related to wound treatment (for example, pressure, flow rate, etc.), data related to the patient, or the like, and transmit the data to the central control monitoring system for processing. The central control monitoring system can determine presence of one or more abnormal operating conditions, such as a leak, canister full, blockage, overpressure, or the like, and instruct the distributed negative pressure wound therapy device to take appropriate action, such as provide an indication, pause therapy, or the like. The central control monitoring system can monitor progress of therapy and make any adjustments to the prescription or delivery of negative pressure wound therapy (such as, change therapy setpoint, switch to continuous or intermittent therapy, switch to a canisterless negative pressure wound therapy device, or the like). The distributed negative pressure wound therapy device can verify integrity of data transmitted to and received from the central control monitoring system.

The distributed negative pressure wound therapy device can switch between different communication protocols or different modes of the same communication protocol, for instance, to conserve power. A lower speed and lower energy consumption communication protocol, such as narrowband internet of things (NB-IoT or LTE NB, for instance, LTE Cat NB1 or NB2), can be used in the default configuration. For instance, the distributed negative pressure wound therapy device can use the lower speed and lower energy consumption protocol when the device is in transit or storage. Being in storage (or in transit) can be detected responsive to lack of movement. One or more motion sensors, such as accelerometers or gyroscopes, can be used to monitor movement or lack of movement. Responsive to detection of being in storage (or in transit), the distributed negative pressure wound therapy device can transmit one or more of location data or power source level via the lower speed and lower energy consumption protocol. Such transmission can be performed periodically.

The distributed negative pressure wound therapy device can switch to a higher speed and higher energy consumption communication protocol, such as LTE-M (for instance, LTE Cat M1), 4G LTE, 5G, 6G, or the like as needed. For example, the device can switch responsive to detection of patient movement or the like, which can indicate that the device is being used to deliver therapy to the patient. Patient movement can be detected, for instance, by one or more motion sensors, such as accelerometers or gyroscopes, of the distributed negative pressure wound therapy device. As another example, the device can switch responsive to detection that provision of negative pressure wound therapy has been activated, which can indicate that the device is being used to deliver therapy to the patient.

Responsive to the detection of being in use to deliver therapy, the distributed negative pressure wound therapy device can transmit one or more therapy parameters via the higher speed and higher energy consumption communication protocol. Such transmission can be performed periodically. As described herein, adjustment of one or more therapy parameters by the central monitoring system can be performed via the higher speed and higher energy consumption communication protocol. The distributed negative pressure wound therapy device can revert to the default configuration, for instance, responsive to no longer detecting patient movement. Such transition can be performed in response to no longer detecting patient movement over a threshold duration of time. The threshold duration of time can be adjusted based on the time of the day. For example, during nighttime, a longer threshold duration of time can be used as compared to daytime. As another example, transmission of certain data that requires higher transmission speed (such as, multimedia data) to the device can be accomplished using the higher speed communication protocol.

Healthcare provider can review data stored by the central control monitoring system and request to adjust one or more therapy parameters (for instance, increase or decrease negative pressure level, decrease negative pressure level, switch from continuous to intermittent mode or vice versa, take a picture or video of the wound, take picture or video of the patient, call the patient, etc.). Healthcare provider can have direct access to a patient via the distributed negative pressure wound therapy device and substantially in real time. Assessments by the healthcare provider can be requested by the central processing system. Healthcare provider can request additional interrogation of the patient by the distributed negative pressure wound therapy system 500.

The system 500 can monitor loss of communication with the distributed negative pressure wound therapy device 110″ and take appropriate remedial actions. Such loss may be due to poor network conditions, problem with the device hardware or software, or the like. Information regarding loss of communication may be available at the central control monitoring system and the device 110″. As a result, performance of one or more remedial actions can be initiated by the central control monitoring system or the device 110″.

Responsive to detecting loss of communication, a mesh network can be utilized to relay one or more commands to the device 110″ via another nearby distributed negative pressure wound therapy device or another computing device, such as the remote computing device 334. Communications over the mesh network can be accomplished via a different communication protocol than the one used by the device 110″ to communicate with the central control monitoring system. Such protocol can be a short range wireless communication protocol, for instance, Bluetooth, BLE, Zigbee, or the like. Advantageously, this can provide redundancy for controlling the device 110″ using another communication protocol (which may use different hardware or software) of the device 110″ even when the device 110″ is unable to directly communicate with the central control monitoring system. One or more commands can include shutting down the device, directing the device to provide negative pressure wound therapy according to a default set of parameters (to ensure essential therapy performance), or the like. In case there is a problem with reaching the central control monitoring system (for instance, due to poor network conditions), another central control monitoring system can be utilized to control the device 110″. Such another central control monitoring system can be local to the device 110″.

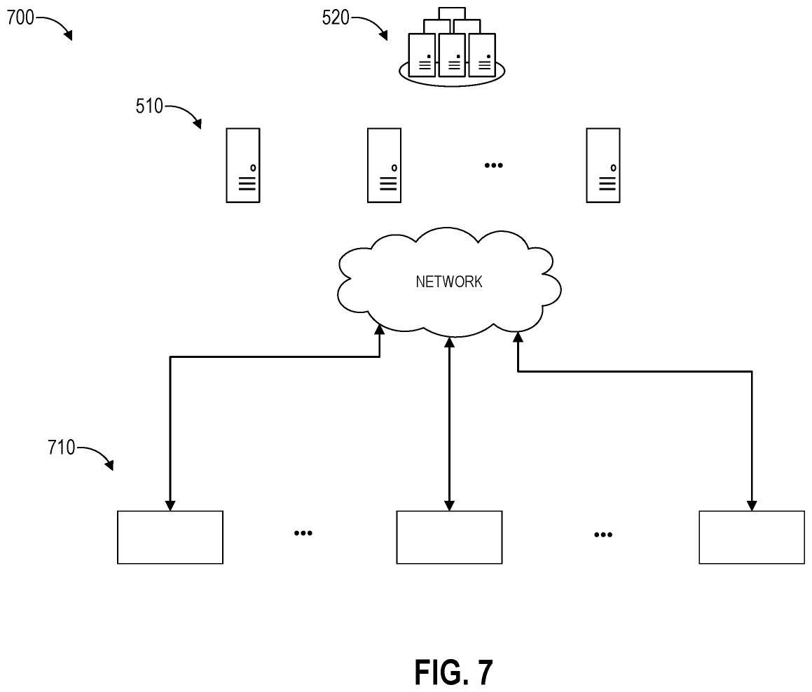

Disclosed implementations are not limited to controlling distributed negative pressure wound therapy devices but can be used for any medical device. FIG. 7 illustrates a distributed medical device system 700. A plurality of medical devices 710, such as one or more negative pressure wound therapy devices (for instance, one or more devices 110″), one or more implantable devices, one or more non-implantable devices, one or more wearable devices, or the like can communicate with the computing devices 510 and 520 as described herein.

As disclosed herein, advantages of the disclosed distributed systems include smaller and lighter negative pressure wound therapy devices, more environmentally friendly devices, devices that be disposed of easier, centralized data collection and control (which can facilitate improved therapy compliance, regulatory compliance, etc.), or the like.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any implantable or non-implantable medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. A negative pressure wound therapy system comprising:
at least one negative pressure wound therapy device configured to provide, via a fluid flow path, negative pressure therapy to a wound of a patient covered by a wound dressing in accordance with at least one parameter, the at least one negative pressure wound therapy device being configured to measure a pressure in the fluid flow path, and the at least one negative pressure wound therapy device being configured to switch between different communication protocols responsive to a detection of movement of the patient; and
one or more remote computing devices configured to:
communicate with the at least one negative pressure wound therapy device via a low latency, high speed communication protocol;
receive the pressure in the fluid flow path from the at least one negative pressure wound therapy device;
based on processing the pressure in the fluid flow path, determine whether an adjustment to the at least one parameter of negative pressure therapy is needed; and
in response to determining that the adjustment to the at least one parameter of negative pressure therapy is needed, communicate to the at least one negative pressure wound therapy device a request to perform the adjustment, wherein, the at least one negative pressure wound therapy device is configured to perform the adjustment in response to receiving the request.

2. (canceled)

3. (canceled)

4. The negative pressure wound therapy system of claim 1, wherein the at least one negative pressure wound therapy device is configured to switch from NB-IoT mode to LTE-M mode responsive to the detection of movement of the patient and switch from LTE-M mode to NB-IoT mode responsive to lack of the detection of movement of the patient.

5. (canceled)

6. (canceled)

7. (canceled)

8. (canceled)

9. (canceled)

10. (canceled)

11. (canceled)

12. A negative pressure wound therapy device comprising:
a negative pressure source configured to provide, via a fluid flow path, negative pressure therapy to a wound of a patient covered by a wound dressing in accordance with at least one parameter of therapy;
a pressure sensor configured to measure a pressure in the fluid flow path; and
an electronic processing circuitry configured to transmit data to and receive data from one or more remote computing devices via a low latency, high speed communication protocol, the electronic processing circuitry being further configured to:
transmit the pressure in the fluid flow path measured by the pressure sensor, thereby causing the one or more remote computing devices to determine an adjustment to the at least one parameter of therapy;
receive from the one or more remote computing devices the adjustment of the at least one parameter of therapy; and
adjust the at least one parameter of therapy using the adjustment.

13. The negative pressure wound therapy device of claim 12, wherein the low latency, high speed communication protocol comprises a 4G LTE or 5G cellular communication protocol.

14. The negative pressure wound therapy device of claim 12, wherein the electronic processing circuitry is configured to switch between different communication protocols responsive to a detection of movement of the patient.

15. The negative pressure wound therapy device of claim 14, wherein the electronic processing circuitry is configured to switch from a low speed, low energy consumption communication protocol to the low latency, high speed communication protocol responsive to the detection of movement of the patient.

16. The negative pressure wound therapy device of claim 14, wherein the electronic processing circuitry is configured to switch from NB-IoT mode to LTE-M mode responsive to the detection of movement of the patient and switch from LTE-M mode to NB-IoT mode responsive to lack of the detection of movement of the patient.

17. The negative pressure wound therapy device of claim 12, wherein adjustment of the at least one parameter of therapy comprises at least one of changing a negative pressure set point, providing indication of an abnormal operating condition, or pausing provision of negative pressure therapy by the negative pressure source.

18. The negative pressure wound therapy device of claim 12, wherein the electronic processing circuitry is configured to receive from the one or more remote computing devices a negative pressure wound therapy prescription and cause the negative pressure source to provide negative pressure in accordance with the negative pressure wound therapy prescription.

19. The negative pressure wound therapy device of claim 18, wherein the electronic processing circuitry is configured to determine a verification of the negative pressure wound therapy prescription by a healthcare provider at the negative pressure wound therapy device prior to causing the negative pressure source to provide negative pressure in accordance with the negative pressure wound therapy prescription.

20. (canceled)

21. A negative pressure wound therapy system comprising:
at least one negative pressure wound therapy device configured to provide, via a fluid flow path, negative pressure therapy to a wound of a patient covered by a wound dressing in accordance with at least one parameter, the at least one negative pressure wound therapy device being configured to measure a pressure in the fluid flow path, and the at least one negative pressure wound therapy device being configured to switch between different communication protocols responsive to a detection of movement of the patient; and
a non-transitory computer readable medium storing instructions that, when executed by one or more processors of one or more remote computing devices, cause the one or more processors to:
communicate with the at least one negative pressure wound therapy device via a low latency, high speed communication protocol;
receive the pressure in the fluid flow path from the at least one negative pressure wound therapy device;
based on processing the pressure in the fluid flow path, determine whether an adjustment to the at least one parameter of negative pressure therapy is needed; and
in response to determining that the adjustment to the at least one parameter of negative pressure therapy is needed, communicate to the at least one negative pressure wound therapy device a request to perform the adjustment, wherein, the at least one negative pressure wound therapy device is configured to perform the adjustment in response to receiving the request.

22. The negative pressure wound therapy system of claim 21, wherein the low latency, high speed communication protocol comprises a 4G LTE or 5G cellular communication protocol.

23. The negative pressure wound therapy system of claim 21, wherein the at least one negative pressure wound therapy device is configured to switch from a low speed, low energy consumption communication protocol to the low latency, high speed communication protocol responsive to the detection of movement of the patient.

24. The negative pressure wound therapy system of claim 21, wherein the at least one negative pressure wound therapy device is configured to switch from NB-IoT mode to LTE-M mode responsive to the detection of movement of the patient and switch from LTE-M mode to NB-IoT mode responsive to lack of the detection of movement of the patient.

25. The negative pressure wound therapy system of claim 21, wherein the adjustment comprises at least one of changing a negative pressure set point, providing indication of an abnormal operating condition, or pausing provision of negative pressure therapy.

26. The negative pressure wound therapy system of claim 21, wherein the instructions further cause the one or more processors to communicate to the at least one negative pressure wound therapy device a negative pressure wound therapy prescription, and wherein the at least one negative pressure wound therapy device is configured to provide negative pressure therapy in accordance with the negative pressure wound therapy prescription.

27. The negative pressure wound therapy system of claim 26, wherein the at least one negative pressure wound therapy device is configured to provide negative pressure therapy in accordance with the negative pressure wound therapy prescription responsive to verification of the negative pressure wound therapy prescription by a healthcare provider at the at least one negative pressure wound therapy device.

28. The negative pressure wound therapy system of claim 21, wherein the at least one negative pressure wound therapy device comprises a plurality of negative pressure wound therapy devices configured to be controlled by the one or more processors.

29. The negative pressure wound therapy system of claim 21, wherein in response to detecting a failure in communication via the low latency, high speed communication protocol, communication with the at least one negative pressure wound therapy device is performed using a different communication protocol via another negative pressure wound therapy device or another computing device different from the one or more remote computing devices.

30. The negative pressure wound therapy system of claim 29, wherein the different communication protocol comprises a short range wireless communication protocol.
